(19) United States
(12) Patent Application Publication
(10) Pub. No.: US 2014/0219938 A1
(43) Pub. Date: Aug. 7, 2014

(54) SUNSCREENS

(71) Applicant: Christine Mendrok-Edinger, Basel (CH)

(72) Inventor: Christine Mendrok-Edinger, Basel (CH)

(73) Assignee: DSM IP ASSETS B.V., Heerlen (NL)

(21) Appl. No.: 14/124,898

(22) PCT Filed: Jun. 6, 2012
(86) PCT No.: PCT/EP2012/060655

§ 371 Date: Mar. 31, 2014

(30) Foreign Application Priority Data
Jun. 8, 2011 (EP) 11169195.2
Jul. 12, 2011 (EP) 11173599.9

(51) Int. Cl. A61K 8/06; A61K 8/44; A61K 8/35; A61Q 17/04; A61K 8/49

(52) U.S. Cl. CPC A61K 8/062; A61Q 17/04; A61K 8/496; A61K 8/35; A61K 8/445
USPC 424/60

(57) ABSTRACT

The present invention relates to a composition for topical application in the form of an oil-in-water (O/W) emulsion comprising in a physiologically acceptable medium at least a dibenzoylmethane derivative, an amino substituted hydroxybenzophenone derivative and a benzotriazol derivative.

The present invention relates to a composition for topical application in the form of an oil-in-water (O/W) emulsion comprising in a physiologically acceptable medium at least a dibenzoylmethane derivative, an amino substituted hydroxybenzophenone derivative and a benzotriazol derivative.

Sun care products have evolved considerably over the years. Earlier formulations were intended to protect the user from UV-B radiation as was once thought that UV-B rays were the most important contributors to wrinkling, skin disease, and skin cancer. However, more recent studies have shown that UV-A radiation is equally or even more important in the development of solar damage and skin diseases, such as lupus erythematosus and melanoma and non-melanoma skin cancers. Thus, today's focus is towards eliminating as much of UVA (320-400 nm) and/or UVB (280-320 nm) light as possible. Consequently, there's a constantly increasing need for sun care products exhibiting high SPF's (Sun Protection Factors) and high UVA protection while being photostable.

However, the so far most commonly used UV-A filter substances (INCI names) butyl methoxy dibenzoylmethane [CAS 70356-09-1] and diethylamino hydroxybenzoyl hexyl benzoate [CAS 302776-68-7] degrade upon irradiation (by cross reaction) when formulated together.

It was therefore the object of the present invention to remedy the disadvantages of the prior art and to develop photostable sunscreen compositions comprising butyl methoxy dibenzoylmethane and diethylamino hydroxybenzoyl hexyl benzoate.

Surprisingly, it has been found that when butyl methoxy dibenzoylmethane and diethylamino hydroxybenzoyl hexyl benzoate are formulated together with specific benzotriazol derivative into O/W emulsions the incompatibility problem can be significantly reduced. This also applies for dibenzoylmethane derivatives and amino substituted hydroxybenzophenone derivatives in general. Furthermore, the SPF of such compositions is increased.

Thus, the invention relates in one aspect to a topical composition in the form of an oil-in-water emulsion comprising a dibenzoylmethane derivative and an amino substituted hydroxybenzophenone derivative, characterized in that at least one benzotriazol derivative of formula (I)

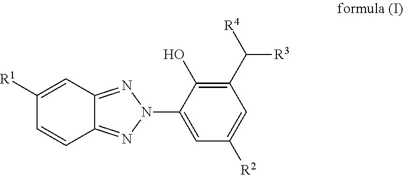

wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl, C₅₋₁₀cycloalkyl, C₁₋₂₀alkoxy or C₅₋₁₀cycloalkoxy; preferably C₅₋₁₅alkyl or C₅₋₁₅alkoxy; and
R⁴ is hydrogen or C₁₋₅alkyl; preferably hydrogen
is present in an amount ranging from 1 to 20 wt.-%, based on the total weight of the composition.

In a particular embodiment the benzotriazol derivative is selected from compounds of formula (I) wherein R¹ and R⁴ are hydrogen, R² is methyl and R³ is C₅₋₁₀alkoxy, such as in particular C₆₋₁₀alkoxy, or C₆cycloalkoxy such as 2,5,5-trimethylhexyloxy, 3,5,5-trimethylhexyloxy, isoamyloxy, 2-ethylhexyloxy or 3,3,5-trimethyl-cyclohexyloxy. Such compounds and their preparation are e.g. disclosed in EP Application No.: 10150832.3 (PCT publication: WO2011/086124). Particularly preferred according to the present invention is a compound of formula (I), wherein R¹ and R⁴ are hydrogen, R² is methyl and R³ is 2-ethylhexyloxy (i.e. 2-(2H-Benzotriazol-2-yl)-6-(2-ethylhexyloxymethyl)-4-methyl-phenol).

In another particular embodiment, the compound of formula (I) is a compound wherein R¹ and R⁴ are hydrogen, R² is methyl and R³ is undecyl (C₁₁H₂₃) which is commercially available as Tinogard TL [INCI Name: Benzotriazolyl Dodecyl p-Cresol; IUPAC Name, 2-(2H-benzotriazol-2-yl)-6-dodecyl-4-methyl-phenol] at BASF SE Ludwigshafen.

Particular suitable dibenzoylmethane derivatives according to the invention encompass 2-methyldibenzoylmethane, 4-methyldibenzoylmethane, 4-isopropyldibenzoylmethane, 4-tert.-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 4-tert.-butyl-4′-methoxydibenzoylmethane, 2-methyl-5-isopropyl-4′-methoxydibenzoylmethane, 2-methyl-5-tert-butyl-4′-methxydibenzoylmethane, 2,4-dimethyl-4′-methoxydibenzoylmethane, 2,6-dimethyl-4-tert-butyl-4′-methoxydibenzoylmethane such as most in particular 4-(tert.-butyl)-4′-methoxydibenzoylmethane (INCI Name: butyl methoxy dibenzoylmethane; IUPAC Name: 1-(4-Methoxyphenyl)-3-(4-tert-butylphenyl)propane-1,3-dione) which is e.g. commercially available as PARSOL® 1789 at DSM Nutritional Products Ltd Kaiseraugst.

Particular suitable amino substituted hydroxybenzophenone derivatives encompass compounds of formula (II)

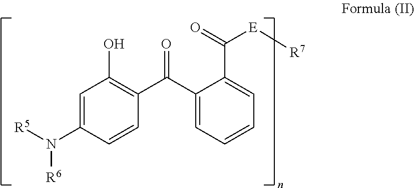

wherein
R⁵ and R⁶ independently of each other are hydrogen; C₁₋₂₀alkyl; C₂₋₂₀alkenyl; C₅₋₁₀cycloalkyl or C₅₋₁₀cycloalkenyl; or R⁵ and R⁶, together with the nitrogen atom they are bound to, form a 5 to 6 membered ring;
n is an integer from 1 or 2;

### E is —O— or —N(R⁸)— and

R⁸ is hydrogen; C₁₋₅alkyl; or C₁₋₅hydroxyalkyl;
with the proviso that
when n=1 then R⁷ is C₁₋₂₀alkyl; C₂₋₂₀alkenyl; C₁₋₅hydroxyalkyl; C₅₋₁₀cycloalkyl; C₅₋₁₀cycloalkeny; C₆₋₁₀aryl; or aralkyl optionally substituted by O, N or S; or a C₁₋₅ aminocarbonyl or alkylcarbonyl radical;
when n=2 then R⁷ is an C₁₋₂₀alkyl; C₅₋₁₀cycloalkyl-; C₂₋₂₀alkenyl- or aryl-diradical or R⁷ with E forms a diradical of formula (III) resulting in a compound of formula (IV)

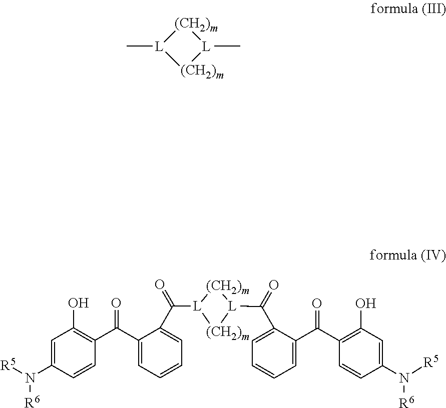

wherein L is N (nitrogen) and
m is an integer between 1 and 3.

A particular suitable amino substituted hydroxybenzophenone derivative according to the present invention is diethylamino hydroxybenzoyl hexyl benzoate [IUPAC Name: Hexyl 2-[4-(diethylamino)-2-hydroxybenzoyl]benzoate] sold under the tradename Uvinul® A plus by BASF SE Ludwigshafen.

Another suitable amino substituted hydroxybenzophenone derivative according to the present invention is 1,1′-(1,4-piperazinediyl)bis[1-[2-[4-(diethylamino)-2-hydroxybenzoyl]phenyl]-methanone [CAS No 919803-06-8] which is e.g. disclosed in WO 2007071584.

Compounds according to formula (II) and their preparation are e.g. disclosed in EP1046391, EP1133980, DE10012408 and WO 2007071584.

The term “C_{x-y}alkyl” as used herein refers to straight-chain or branched alkyl radicals with x to y carbon atoms such as e.g. methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methyl pentyl, 3-methylpentyl, 4-methylpentyl, 1,1-dimethylpropyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyle, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl without being limited thereto.

The term C₂₋₂₀alkenyl as used herein refers to straight-chain or branched alkenyl radicals, such as e.g. 2-propen-2-yl, 2-propen-3-yl, 3-buten-3-yl, 3-buten-4-yl, 4-penten-4-yl, 4-penten-5-yl, (3-methyl)-penta-2,4-dien-4-yl, (3-methyl)-penta-2,4-dien-5-yl or 11-dodecen-11-yl.

The term “C_{x-y}cycloalkyl” denotes to unsubstituted or C₁₋₁₀alkyl, in particular C₁₋₅alkyl (mono- or poly-)substituted cyclic, bicyclic or tricyclic hydrocarbon residues with x to y carbon atoms such as in particular cyclopentyl, cyclohexyl, cycloheptyl or decahydronaphtyl. Preferably, C₅₋₁₀cycloalkyl denotes to unsubstituted or C₁₋₂alkyl (mono- or poly-)substituted cyclopentyl, cyclohexyl or cycloheptyl such as in particular to unsubstituted or methyl substituted cyclohexyl such as most in particular to cyclohexyl or 3,3,5-trimethyl-cyclohexyl.

The term C₅₋₁₀cycloalkenyl as used herein refers to cycloalkenyl radicals with one or more double bonds such as e.g. cyclopentenyl, cyclopentadienyl, cyclohexenyl, 1,3-cyclohexadienyl, 1,4-cyclohexadienyl, cycloheptenyl, cycloheptatrienyl, cyclooctenyl, 1,5-cyclooctadienyl, cyclooctatetraenyle, cyclononenyl or cyclodecenyl.

The terms “C_{x-y}alkoxy” respectively “C_{x-y}cycloalkoxy” as used herein denotes to linear or branched alkoxy-, respectively unsubstituted or (mono- or poly-)substituted cycloalkoxy radicals having from x to y carbon atoms such as e.g. methoxy, ethoxy, propoxy, butyloxy or pentyloxy, 2,5,5-trimethylhexyloxy, 3,5,5-trimethylhexyloxy, isoamyloxy, 2-ethylhexyloxy or 3,3,5-trimethyl-cyclohexyloxy.

The term C₆₋₁₀aryl refers e.g. to naphthyl or phenyl radicals, preferably to phenyl.

Suitable examples of 5 or 6-membered rings formed by R⁵ and R⁶ with the nitrogen atom encompass in particular pyrrolidin or piperidin.

The term “topical” is understood here to mean external application to keratinous substances, which are in particular the skin, scalp, eyelashes, eyebrows, nails, mucous membranes and hair.

As the compositions according to the invention are intended for topical application, they comprises a physiologically acceptable medium, that is to say a medium compatible with keratinous substances, such as the skin, mucous membranes, keratinous fibres, such as preferably the skin. In particular the physiologically acceptable medium is a cosmetically acceptable carrier.

The term cosmetically acceptable carrier refers to all carriers and/or excipients and/or diluents conventionally used in cosmetic compositions.

The amount of the at least one benzotriazol derivative of formula (I) in the compositions according to the invention is preferable selected in the range of 2 to 15 wt.-%, in particular in the range of 4 to 10 wt.-%, most particular in the range of 4 to 8 wt.-% based on the total weight of the composition.

The amount of the dibenzoylmethane derivative such as in particular of butyl methoxydibenzoylmethane in the compositions according to the invention is preferable selected in the range of 2 to 8 wt.-%, in particular in the range of 3 to 6 wt.-%, most particular in the range of 3 to 5 wt.-%, based on the total weight of the composition.

The amount of the amino substituted hydroxybenzophenone derivative such as in particular of diethylamino hydroxybenzoyl hexyl benzoate in the compositions according to the invention is preferable selected in the range of 2 to 8 wt.-%, in particular in the range of 3 to 6 wt.-%, most particular in the range of 3 to 5 wt.-%, based on the total weight of the composition.

The topical composition in the form of an oil-in-water emulsion according to the present invention comprises an oily phase dispersed in an aqueous phase in the presence of an O/W emulsifier. The preparation of such oil-in-water emulsions is well known to a person skilled in the art and illustrated in the examples.

Suitable O/W emulsifiers are oil-in-water (O/W) or silicone-in-water (Si/W)-emulsifiers selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, glycerylstearatcitrate, glycerylstearate (self emulsifying), stearic acid, salts of stearic acid, polyglyceryl-3-methylglycosedistearate. Further suitable emulsifiers are phosphate esters and the salts thereof such as cetyl phosphate (Amphisol® A), diethanolamine cetyl phosphate (Amphisol DEA), potassium cetyl phosphate (Amphisol® K), sodiumcetearylsulfat, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

The at least one O/W emulsifier is preferably used in an amount of 0.1 to 10 wt.-%, in particular in the range of 0.5 to 6 wt.-%, such as more in particular in the range of 0.5 to 5 wt.-% such as most in particular in the range of 1 to 4 wt.-%, with respect to the total weigh of the composition.

Particular suitable O/W emulsifiers according to the present invention encompass phosphate esters emulsifiers of formula (IV)

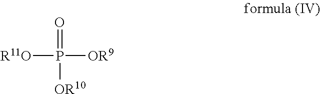

wherein R⁹, R¹⁰ and R¹¹ may be hydrogen; an alkyl of from 1 to 22 carbons, preferably from 12 to 18 carbons; or an alkoxylated alkyl having from 1 to 22 carbons, preferably from 12 to 18 carbons and having 1 or more, preferably from 2 to 25, most preferably 2 to 12, moles ethylene oxide; with the provision that at least one of R⁹, R¹⁰ and R¹¹ is an alkyl or alkoxylated alkyl as previously defined but having at least 6 alkyl carbons in said alkyl or alkoxylated alkyl group.

Monoesters in which R⁹ and R¹⁰ are hydrogen and R¹¹ is selected from alkyl groups of 10 to 18 carbons and alkoxylated fatty alcohols of 10 to 18 carbons and 2 to 12 moles ethylene oxide are preferred. Among the preferred phosphate ester emulsifiers are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, and Deceth-6 Phosphate. Particular preferred phosphate ester surfactants according to the invention are cetyl phosphate, potassium cetyl phosphate and/or DEA cetyl phosphate. A particular advantageous phosphate ester emulsifier according to the invention is Potassium cetyl phosphate e.g. commercially available as Amphisol® K at DSM Nutritional Products Ltd Kaiseraugst.

Further suitable O/W emulsifiers are polyethyleneglycol (PEG) esters or diesters such as e.g. [INCI Names] PEG-100 Stearate, PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate.

Particularly preferred according to the present invention is PEG-100 Stearate e.g. sold under the tradename Myrj S100 by Croda or in combination with glyceryl stearate under the tradename Arlacel™ 165 (INCI Glyceryl Stearate (and) PEG-100 Stearate) by Croda.

Other suitable O/W emulsifiers are anionic polymeric O/W emulsifiers such as e.g. a block copolymer consisting of a poly acrylic acid such as e.g. hydrophobically modified polyacrylic acid (i.e. Acrylates/C10-30 Alkyl Acrylate Crosspolymers) produced by Noveon Inc and sold as Pemulen® TR-1 or TR2.

The topical compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 5 wt.-%, such as most in particular in the range of 1 to 3 wt.-%, based on the total weight of the composition. Particular suitable co-surfactants are selected from the list of alkyl alcohols such as cetyl alcohol (Lorol C16, Lanette 16) cetearyl alcohol (Lanette 0), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof. In case the O/W emulsifier is a phosphate ester emulsifier such as in particular potassium cetyl phosphate or a polyethyleneglycol (PEG) ester or diester stearate and/or stearate emulsifier such as Glyceryl Stearate (and) PEG-100 Stearate then preferably the co-surfactant is cetearyl alcohol (Lanette 0).

The aqueous phase of the composition according to the invention comprises water and optionally one or more compounds which are miscible with water or at least partially miscible with water, such as polyols or lower C₂ to C₈ monoalcohols, such as ethanol and isopropanol. The term “ambient temperature” should be understood as meaning a temperature of approximately 25° C. at standard atmospheric pressure (760 mmHg).

The term “polyol” should be understood as meaning any organic molecule comprising at least two free hydroxyl groups. Mention may be made, as polyols, for example, of glycols, such as butylene glycol, propylene glycol, isoprene glycol, glycerol and polyethylene glycols, such as PEG-8, sorbitol or sugars, such as glucose.

The aqueous phase can also comprise any normal water-soluble or water-dispersible additive well known to a person skilled in the art.

The aqueous phase can represent from 60 to 98% by weight, preferably from 65 to 95 wt.-%, better still from 70 to 90 wt.-% and even better still from 70 to 85 wt.-%, based on the total weight of the composition.

The water-miscible compound or compounds, such as polyols and lower alcohols, can be present in an amount ranging from 0 to 30 wt.-%, based on the total weight of the composition, in particular from 0.1 to 30 wt.-% and better still in an amount ranging from 1 to 20 wt.-%.

The nature of the oily phase of the emulsion according to the invention is not critical. The oily phase is a fatty phase comprising at least one fatty substance chosen from fatty substances which are liquid at ambient temperature (20-25° C.) or volatile or non-volatile oils of vegetable, mineral or synthetic origin, and their mixtures. These oils are physiologically acceptable.

The oily phase can also comprise any normal fat-soluble or fat-dispersible additive well known to a person skilled in the art.

It can in particular comprise other fatty substances, such as waxes, pasty compounds, fatty alcohols or fatty acids. The oily phase comprises at least one oil, more particularly at least one cosmetic oil. The term “oil” is understood to mean a fatty substance which is liquid at ambient temperature (25° C.).

The dibenzoylmethane derivative, the amino substituted hydroxybenzophenone derivative as well as the benzotriazol derivative are advantageously incorporated into the oily phase.

According to one embodiment, the compositions according to the invention comprises less than 60 wt.-% of oils, with respect to the total weight of the composition, preferably less than 50 wt.-% and better still less than 40 wt.-%.

The compositions of the invention are advantageously prepared according to a process in which the oily phase, comprising the oils and optionally the other fatty substances, is emulsified in the aqueous phase with stirring. Stirring is preferably carried out with a magnetic bar, a stirrer or any other stirring system such as a homogenizer at a temperature which can range from 20° C. to 90° C. such as at 40° C. to 80° C.

The compositions according to the invention can be provided, for example, in all the formulation forms for O/W emulsions, for example in the form of serum, milk, lotion or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

Particular preferred topical composition according to the invention are characterized in that the dibenzoylmethane derivative is butyl methoxydibenzoylmethane, the aminobenzophenone derivative is diethylamino hydroxybenzoyl hexyl benzoate and the benzotriazol derivative is benzotriazolyl dodecyl p-cresol or 2-(2H-Benzotriazol-2-yl)-6-(2-ethylhexyloxymethyl)-4-methyl-phenol).

Another advantageous embodiment of the invention relates to a cosmetic composition for topical application in the form of an oil-in-water emulsion comprising an oily phase dispersed in an aqueous phase in the presence of an O/W emulsifier, characterized in that it further comprises at least

(a.) butyl methoxydibenzoylmethane in an amount of 2 to 8 wt.-%, preferably in an amount of 3 to 6 wt.-%, most preferably of 3 to 5 wt.-%,
(b.) diethylamino hydroxybenzoyl hexyl benzoate in an amount of 2 to 8 wt.-%, preferably in an amount of 3 to 6 wt.-%, most preferably of 3 to 5 wt.-% and
(c.) benzotriazolyl dodecyl p-cresol in an amount of 2 to 15 wt.-%, preferably of 4 to 10 wt.-%, most preferably of 4 to 8 wt.-%.

Particular advantageous are compositions according to the present invention comprising butyl methoxydibenzoylmethane and diethylamino hydroxybenzoyl hexyl benzoate in an amount, independently of each other, of 3 to 5 wt.-% and benzotriazolyl dodecyl p-cresol in an amount of 4 to 8 wt.-%. Furthermore it is particularly advantageous if the O/W emulsifier is a phosphate ester emulsifier as defined above such as particularly potassium cetyl phosphate as this leads to particularly good results.

In another advantageous embodiment of the invention relates to a cosmetic composition for topical application in the form of an oil-in-water emulsion comprising an oily phase dispersed in an aqueous phase in the presence of an O/W emulsifier, characterized in that it further comprises at least

(a.) butyl methoxydibenzoylmethane in an amount of 2 to 8 wt.-%, preferably in an amount of 3 to 6 wt.-%, most preferably of 3 to 5 wt.-%,
(b.) diethylamino hydroxybenzoyl hexyl benzoate in an amount of 2 to 8 wt.-%, preferably in an amount of 3 to 6 wt.-%, most preferably of 3 to 5 wt.-% and
(c.) 2-(2H-Benzotriazol-2-yl)-6-(2-ethylhexyloxymethyl)-4-methyl-phenol) in an amount of 2 to 15 wt.-%, preferably of 4 to 10 wt.-%, most preferably of 4 to 8 wt.-%.

Particular advantageous are compositions according to the present invention comprising butyl methoxydibenzoylmethane and diethylamino hydroxybenzoyl hexyl benzoate, independently of each other, in an amount of 3 to 5 wt.-% and 2-(2H-Benzotriazol-2-yl)-6-(2-ethylhexyloxymethyl)-4-methyl-phenol) in an amount of 4 to 8 wt.-%. Furthermore it is particularly advantageous if the O/W emulsifier is a phosphate ester emulsifiers as defined above such as potassium cetyl or a PEG ester or diester such as particularly Glyceryl Stearate (and) PEG-100 Stearate.

In another particular embodiment the compositions according to the present invention are substantially free of a polyglycerol based UV-filter such as e.g. disclosed in [EP Application No's] EP09178503.0, EP09178501.4, EP09178502.2 EP09178495.9, EP09178506.3, EP09178505.5 or EP10150832.3 which are obtainable by a process comprising the steps of ring-opening polymerization of x mol equivalents of glycidol using 1 mol equivalent of a polyol starter unit with y mol equivalents hydroxyl-groups, followed by block copolymerization with z X (x+y) mole equivalents of propylene oxide to form a hyperbranched polyether-polyol backbone carrying (x+y) mol equivalents hydroxyl-groups followed by partial or total esterification, respectively partial or total etherification of the hydroxyl groups with a UV-light absorbing chromophore such as particularly with p-dimethylamino benzoic acid, 3-[1-(4-Hydroxymethyl-phenyl)-meth-(E)-ylidene]-1,7,7-trimethyl-bicyclo[2.2.1]heptan-2-one, 2-(4-diethylamino-2-hydroxybenzoyl)benzoic acid, p-alkoxycinnamic acid, 2-cyano-3,3-diphenylacrylic acid as well as mixtures thereof and wherein x is an integer selected in the range from 3-16, y is an integer selected in the range from 1-6, and z is an integer selected in the range from 0-10.

Finally, a subject-matter of the invention is a method for the cosmetic treatment of keratinous substances such as in particular the skin, characterized in that a composition as defined above is applied to the said keratinous substances such as in particular to the skin. The method is in particular suitable to protect the skin against the adverse effects of UV-A and/or UV-B radiation such as in particular sun-burn and/or photoageing.

In accordance with the present invention, the compositions according to the invention may comprise further ingredients and/or additives such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those for make-up products changing the skin color or e.g. those suited for providing a photoprotective effect by physically blocking out UV radiation, or any other ingredients/additives usually formulated into cosmetic compositions. Such cosmetic ingredients commonly used in the skin care industry, which are suitable for use in the compositions of the present invention, are e.g. described in the CTFA Cosmetic Ingredient Handbook, Second Edition (1992), The Cosmetic, Toiletry and Fragrance Association, Inc. without being limited thereto.

The necessary amounts of the cosmetic and dermatological adjuvants and additives can—based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH, Triethanolamine (TEA Care), Tromethamine (Trizma Base) and Aminomethyl Propanol (AMP-Ultra PC 2000) according to standard methods in the art.

In a further embodiment, the invention relates to the use of a benzotriazol derivative with all the definitions and preferences as given above for reducing the photochemical cross reaction of a dibenzoylmethane derivative and an amino substituted hydroxybenzophenone derivative in the presence of each other in an oil-in water emulsion.

Finally, the invention relates to a method of inhibiting the photochemical cross reaction of a dibenzoylmethane derivative and an amino substituted hydroxybenzophenone derivative in the presence of each other in an oil-in water emulsion, said method comprising the step of incorporating into said composition at least one benzotriazol derivative of formula (I), with all definitions and preferences as given above and observing or appreciating the result.

The following examples are provided to further illustrate the compositions and effects of the present invention. These examples are illustrative only and are not intended to limit the scope of the invention in any way.

### EXAMPLE

The following formulations were prepared according to standard methods known in the way.

TABLE 1
O/W Amphisol ® K formulations

| | | Wt.-% | | | | |
|---|---|---|---|---|---|---|
| Trade Name | INCI Name | 1A | 1B | 1C | 1D | 1E |
| Phenonip ® | Phenoxyethanol, Methylparabene, | 0.80 | 0.80 | 0.80 | 0.80 | 0.80 |
| | Ethylparabene, Propylparabene, | | | | | |
| | Butylparabene, Isopbutylparabene | | | | | |
| Keltrol ® CG-T | Xanthan Gum | 0.30 | 0.30 | 0.30 | 0.30 | 0.30 |
| Finsolv ® TN | C12-15 Alkyl Benzoate | 30.00 | 30.00 | 30.00 | 30.00 | 30.00 |
| Amphisol ® K | Potassium Cetyl Phosphate | 5.00 | 5.00 | 5.00 | 5.00 | 5.00 |
| Lanette ® O | Cetearyl Alcohol | 1.50 | 1.50 | 1.50 | 1.50 | 1.50 |
| Tinogard ® TL | Benzotriazolyl dodecyl p-Cresol | | | 4.00 | 8.00 | |
| | 2-(2H-Benzotriazol-2-yl)-6-(2- | | | | | 8.00 |
| | ethylhexyloxymethyl)-4-methyl- | | | | | |
| | phenol (Chemical name)* | | | | | |
| PARSOL ® 1789 | Butyl methoxy dibenzoylmethane | | 4.00 | 4.00 | 4.00 | 4.00 |
| Uvinul ® A Plus | Diethylamino Hydroxybenzoyl | 4.00 | 4.00 | 4.00 | 4.00 | 4.00 |
| | Hexyl Benzoate | | | | | |

| | | |
|---|---|---|
| Water | Aqua | Ad 100 |

*benzotriazol derivative of formula (I): 2-(2H-Benzotriazol-2-yl)-6-(2-ethylhexyloxymethyl)-4-methyl-phenol (i.e. a compound of formula (I), wherein R¹ and R⁴ are hydrogen, R² is methyl and R³ is 2-ethylhexyloxy)

TABLE 2
O/W Arlacel ® 165 formulations

| | | Wt.-% | | | | |
|---|---|---|---|---|---|---|
| Trade Name | INCI Name | 2A | 2B | 2C | 2D | 2E |
| Phenonip ® | Phenoxyethanol, Methylparabene, | 0.80 | 0.80 | 0.80 | 0.80 | 0.80 |
| | Ethylparabene, Propylparabene, | | | | | |
| | Butylparabene, Isopbutylparabene | | | | | |
| Keltrol ® CG-T | Xanthan Gum | 0.30 | 0.30 | 0.30 | 0.30 | 0.30 |
| Finsolv ® TN | C12-15 Alkyl Benzoate | 30.00 | 30.00 | 30.00 | 30.00 | 30.00 |
| Arlacel ® 165 | Glyceryl Stearate, PEG-100 | 5.00 | 5.00 | 5.00 | 5.00 | 5.00 |
| | Stearate | | | | | |
| Lanette O | Cetearyl Alcohol | 1.50 | 1.50 | 1.50 | 1.50 | 1.50 |
| Tinogard ® TL | Benzotriazolyl dodecyl p-Cresol | | | 4.00 | 8.00 | |
| | 2-(2H-Benzotriazol-2-yl)-6-(2- | | | | | 8.00 |
| | ethylhexyloxymethyl)-4-methyl- | | | | | |
| | phenol (Chemical name)* | | | | | |
| PARSOL ® 1789 | Butyl methoxy dibenzoylmethane | | 4.00 | 4.00 | 4.00 | 4.00 |
| Uvinul ® A Plus | Diethylamino Hydroxybenzoyl | 4.00 | 4.00 | 4.00 | 4.00 | 4.00 |
| | Hexyl Benzoate | | | | | |

| | | |
|---|---|---|
| Water | Aqua | Ad 100 |

*benzotriazol derivative of formula (I): 2-(2H-Benzotriazol-2-yl)-6-(2-ethylhexyloxymethyl)-4-methyl-phenol (i.e. a compound of formula (I), wherein R¹ and R⁴ are hydrogen, R² is methyl and R³ is 2-ethylhexyloxy)

TABLE 3
O/W Pemulen ® TR 2 formulations

| | | 3A | 3B | 3C | 3D |
|---|---|---|---|---|---|
| Trade Name | INCI Name | Wt.-% | | | |
| Phenonip ® | Phenoxyethanol, | 0.80 | 0.80 | 0.80 | 0.80 |
| | Methylparabene, | | | | |
| | Ethylparabene, | | | | |
| | Propylparabene, | | | | |
| | Butylparabene, | | | | |
| | Isopbutylparabene | | | | |
| Keltrol ® CG-T | Xanthan Gum | 0.30 | 0.30 | 0.30 | 0.30 |
| Finsolv TN | C12-15 Alkyl Benzoate | 30.00 | 30.00 | 30.00 | 30.00 |
| Pemulen ® TR 2 | Acrylates/C10-30 Alkyl | 0.40 | 0.40 | 0.40 | 0.40 |
| | Acrylate Crosspolymer | | | | |
| Tinogard ® TL | Benzotriazolyl dodecyl | | | 4.00 | 8.00 |
| | p-Cresol | | | | |
| PARSOL ®1789 | Butyl methoxy | | 4.00 | 4.00 | 4.00 |
| | dibenzoylmethane | | | | |
| Uvinul ® A Plus | Diethylamino | 4.00 | 4.00 | 4.00 | 4.00 |
| | Hydroxybenzoyl Hexyl | | | | |
| | Benzoate | | | | |
| TEA Care | Triethanolamine | 0.40 | 0.40 | 0.40 | 0.40 |

| | | |
|---|---|---|
| Water | Aqua | Ad 100 |

TABLE 4
W/O Dehymuls ® PGPH formulations (Reference)

| | | Wt.-% | | | | |
|---|---|---|---|---|---|---|
| Trade Name | INCI Name | 4A | 4B | 4C | 4D | 4E |
| Finsolv ® TN | C12-15 Alkyl Benzoate | 30.00 | 30.00 | 30.00 | 30.00 | 30.00 |
| Dehymuls ® PGPH | Polyglyceryl-2 | 6.00 | 6.00 | 6.00 | 6.00 | 6.00 |
| | Dipolyhydroxystearate | | | | | |
| Keltrol ® CG-T | Xanthan Gum | 0.30 | 0.30 | 0.30 | 0.30 | 0.30 |
| PARSOL ® 1789 | Butyl methoxy | | 4.00 | 4.00 | 4.00 | 4.00 |
| | dibenzoylmethane | | | | | |
| Tinogard ® TL | Benzotriazolyl dodecyl p- | | | 4.00 | 8.00 | |
| | Cresol | | | | | |
| | 2-(2H-Benzotriazol-2-yl)-6-(2- | | | | | 8.00 |
| | ethylhexyloxymethyl)-4-methyl- | | | | | |
| | phenol (Chemical name) | | | | | |
| Uvinul ® A Plus | Diethylamino Hydroxybenzoyl | 4.00 | 4.00 | 4.00 | 4.00 | 4.00 |
| | Hexyl Benzoate | | | | | |
| Magnesiumsulfat | Magnesium Sulfate | 0.50 | 0.50 | 0.50 | 0.50 | 0.50 |
| Heptahydrat | | | | | | |

| | | |
|---|---|---|
| Water | Aqua | Ad 100 |

### Photostability Test Method:

Photostability after irradiation has been tested in analogy to the method described by H. Berset et (G. Berset, H. Gozenbach, R. Christ, R. Martin, A. Deflandre, R. E. Mascotto, J. D. R. Jolley, W. Lowell, R. Pelzer and T. Stiehm, Proposed protocol for determination of photostability. Part I: cosmetic UV filters, Int. J. Cosmet. Sci. 18 (1996), pp. 167-177 (1996)): 1 mg/cm² of the respective formulation has been distributed on glass slides and the samples exposed to 25 MED solar simulated light under an Atlas SunTester XLS+ with an UV irradiance of 64 W/cm² at a temperature of 35° C. The dried film has been immersed in 50 ml of Methanol under treatment in an ultrasonic water bath. Then the UV filter concentration has been determined by HPLC (G. Berset et. al. 1996). The results are outlined in table 5.

TABLE 5
Photostability test results

| | | Amount [wt.-%] | | | | Recovery [%] | |
|---|---|---|---|---|---|---|---|
| No | Emulsifier | (I)* | (II)* | (III)* | (IV)* | (I)* | (II)* |
| 1A (Reference) | Amphisol ® K (O/W emulsifier) | 4 | — | — | — | 100 | — |
| 1B (Reference) | | 4 | 4 | — | — | 47 | 59 |
| 1C | | 4 | 4 | 4 | — | 78 | 80 |
| 1D | | 4 | 4 | 8 | — | 100 | 100 |
| 1E | | 4 | 4 | — | 8 | 83 | 84 |
| 2A (Reference) | Arlacel ® 165 (O/W emulsifier) | 4 | — | — | — | 93 | — |
| 2B (Reference) | | 4 | 4 | — | — | 61 | 63 |
| 2C | | 4 | 4 | 4 | — | 85 | 90 |
| 2D | | 4 | 4 | 8 | | 93 | 91 |
| 2 E | | 4 | 4 | | 8 | 85 | 85 |
| 3A (Reference) | Permulen ® TR2 (O/W emulsifier) | 4 | — | — | — | 97 | — |
| 3B (Reference) | | 4 | 4 | — | — | 54 | 62 |
| 3C | | 4 | 4 | 4 | — | 72 | 77 |
| 3D | | 4 | 4 | 8 | — | 85 | 91 |
| 4A (Reference) | Dehymuls ® PGPH (W/O emulsifier) | 4 | — | — | — | 77 | — |
| 4B (Reference) | | 4 | 4 | — | — | 57 | 59 |
| 4C (Reference) | | 4 | 4 | 4 | — | 56 | 61 |
| 4D (Reference) | | 4 | 4 | 8 | — | 70 | 71 |
| 4E (Reference) | | 4 | 4 | — | 8 | 61 | 55 |

*(I) = amino substituted hydroxybenzophenone derivative: Uvinul A ® plus by BASF
(II) = dibenzoylmethane derivative: PARSOL ® 1789 by DSM nutritional Product
(III) = benzotriazol derivative of formula (I): Tinogard ® TL by BASF
(IV) = benzotriazol derivative of formula (I): 2-(2H-Benzotriazol-2-yl)-6-(2-ethylhexyloxymethyl)-4-methyl-phenol (i.e. a compound of formula (I), wherein R¹ and R⁴ are hydrogen, R² is methyl and R³ is 2-ethylhexyloxy)

As can be retrieved from the table the use of an O/W emulsifiers (=>O/W emulsion) lead to a significant better photostability compared to the use of a W/O emulsifier (=>W/O emulsion).

Furthermore, it can be retrieved that alky phosphates such as Amphisol K as well as a Glyceryl Stearate (and) PEG-100 Stearate emulsifier (Arlacel 165) lead to even better results whereas the alkyl phosphate emulsifier yields the best overall photostability for Uvinul® A plus and Parsol® 1789.

1. A topical composition in the form of an oil-in-water emulsion comprising a dibenzoylmethane derivative and an amino substituted hydroxybenzophenone derivative, characterized in that at least one benzotriazol derivative of formula (I) 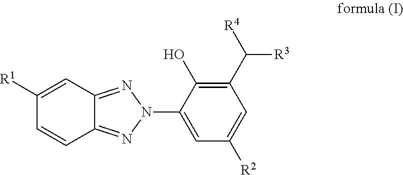
wherein
R¹ is hydrogen; C₁₋₅alkyl; C₁₋₅alkoxy or halogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl;
R³ is C₁₋₂₀alkyl, C₅₋₁₀cycloalkyl, C₁₋₂₀alkoxy or C₅₋₁₀cycloalkoxy; and
R⁴ is hydrogen or C₁₋₅alkyl;
is present in an amount ranging from 1 to 20 wt.-%, based on the total weight of the composition.

2. A topical composition according to claim 1, characterized in that the at least one benzotriazol derivative is selected from compounds of formula (I) wherein R¹ and R⁴ are hydrogen, R² is methyl and R³ is 2,5,5-trimethylhexyloxy, 3,5,5-trimethylhexyloxy, isoamyloxy, 2-ethylhexyloxy, 3,3,5-trimethyl-cyclohexyloxy or undecyl.

3. A topical composition according to claim 1, characterized in that the dibenzoylmethane derivative is butyl methoxydibenzoylmethane.

4. A topical composition according to claim 1, characterized in that the amino substituted hydroxybenzophenone derivative is a compound of formula (II) 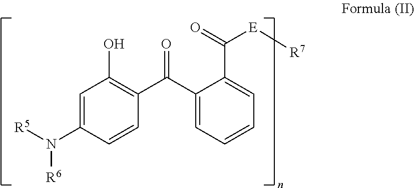
wherein
R⁵ and R⁶ independently of each other are hydrogen; C₁-C₂₀alkyl; C₂-C₂₀alkenyl; C₅₋₁₀cycloalkyl or C₅-C₁₀cycloalkenyl; or R⁵ and R⁶, together with the nitrogen atom they are bound to, form a 5 to 6 membered ring;
n is an integer from 1 or 2;
E is —O— or —N(R⁸)— and
R⁸ is hydrogen; C₁-C₅alkyl; or C₁-C₅hydroxyalkyl;
with the proviso that
when n=1 then R⁷ is C₁-C₂₀alkyl; C₂-C₂₀alkenyl; C₁-C₅hydroxyalkyl; C₅-C₁₀cycloalkyl; C₅-C₁₀cycloalkeny; C₆₋₁₀aryl; or aralkyl optionally substituted by O, N or S; or a C₁-C₅ aminocarbonyl or alkylcarbonyl radical;
when n=2 then R⁷ is an C₁-C₂₀alkyl; C₅-C₁₀cycloalkyl-; C₂-C₂₀alkenyl- or aryl-diradical or R⁷ with E forms a diradical of formula (III) 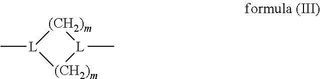
wherein L is N (nitrogen) and
m is an integer between 1 and 3.

5. A topical composition according to claim 1, characterized in that the amino substituted hydroxybenzophenone derivative is diethylamino hydroxybenzoyl hexyl benzoate or 1,1′-(1,4-piperazinediyl)bis[1-[2-[4-(diethylamino)-2-hydroxybenzoyl]phenyl]-methanone.

6. A topical composition according to claim 1, characterized in that the dibenzoylmethane derivative is butyl methoxydibenzoylmethane, the aminobenzophenone derivative is diethylamino hydroxybenzoyl hexyl benzoate and the benzotriazol derivative is benzotriazolyl dodecyl p-cresol or 2-(2H-Benzotriazol-2-yl)-6-(2-ethyl hexyloxymethyl)-4-methyl-phenol).

7. A topical composition according to claim 1, characterized in that the amount of the dibenzoylmethane derivative and of the aminobenzophenone derivative is, independently of each other, selected in the range of 2 to 8 wt.-%, based on the total weight of the composition.

8. A topical composition according to claim 7, characterized in that the amount of the dibenzoylmethane derivative and of the aminobenzophenone derivative is, independently of each other, selected in the range of 3 to 5 wt.-% and the amount of the benzotriazol derivative is selected in the range of 4 to 10 wt.-%, based on the total weight of the composition.

9. A topical composition according to claim 1, characterized in that the oil-in-water emulsifier is selected from the group consisting of phosphate ester, polyethyleneglycol (PEG) ester or diester and/or polymeric oil-in-water emulsifiers.

10. The topical composition according to claim 9, characterized in that the oil-in-water emulsifier is potassium cetyl phosphate.

11. The topical composition according to claim 9, characterized in that the oil-in-water emulsifier is Glyceryl Stearate (and) PEG-100 Stearate.

12. The topical composition according to claim 1, characterized in that the amount of O/W emulsifier is selected in the range of 0.5-5 wt.-%, based on the total weight of the composition.

13. The topical composition according to claim 1, characterized in that the composition comprises at least one co-surfactant in an amount selected in the range of 0.1 to 10 wt.-%, based on the total weight of the composition.

14. Use of a benzotriazol derivative of formula (I) 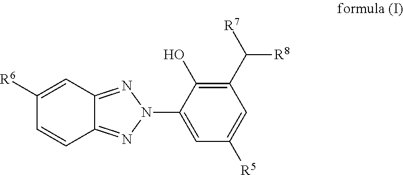
wherein
R⁵ is hydrogen; C₁₋₂₀alkyl; C₁₋₅alkoxy; C₁₋₅alkoxycarbonyl; C₃₋₁₀cycloalkyl; C₆₋₁₀aryl or aralkyl;
R⁶ is hydrogen; C₁₋₅alkyl; C₁₋₅alkoxy or halogen;
R⁷ is hydrogen or C₁₋₅alkyl; and
R⁸ is C₁₋₃₀alkyl, C₃₋₁₀cycloalkyl, C₁₋₃₀alkoxy or C₅₋₁₀cycloalkoxy.
for reducing the photochemical cross reaction of a dibenzoylmethane derivative and an amino substituted hydroxybenzophenone derivative in the presence of each other in an oil-in water emulsion.

15. A method of inhibiting the photochemical cross reaction of a dibenzoylmethane derivative and an amino substituted hydroxybenzophenone derivative in the presence of each other in an oil-in water emulsion said method comprising the step of incorporating into said composition at least one benzotriazol derivative of formula (I), 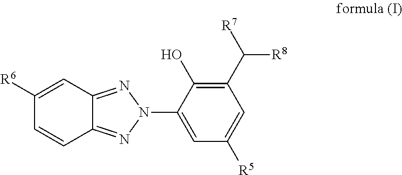
wherein
R⁵ is hydrogen; C₁₋₂₀alkyl; C₁₋₅alkoxy; C₁₋₅alkoxycarbonyl; C₃₋₁₀cycloalkyl; C₆₋₁₀aryl or aralkyl;
R⁶ is hydrogen; C₁₋₅alkyl; C₁₋₅alkoxy or halogen;
R⁷ is hydrogen or C₁₋₅alkyl; and
R⁸ is C₁₋₃₀alkyl, C₃₋₁₀cycloalkyl, C₁₋₃₀alkoxy or C₅₋₁₀cycloalkoxy.
and observing or appreciating the result.
